(19) United States
(12) Patent Application Publication
(10) Pub. No.: US 2011/0077524 A1
(43) Pub. Date: Mar. 31, 2011

(54) ULTRASONIC DIAGNOSTIC APPARATUS AND ULTRASONIC CONTRAST IMAGING METHOD

(76) Inventors: Mitsuhiro Oshiki, Tokyo (JP); Hiroshi Masuzawa, Tokyo (JP)

(21) Appl. No.: 12/994,984

(22) PCT Filed: May 20, 2009
(86) PCT No.: PCT/JP2009/059241

§ 371 Date: Nov. 29, 2010

(30) Foreign Application Priority Data
May 29, 2008 (JP) 2008-140692

(51) Int. Cl. A61B 8/14; A61B 8/00

(52) U.S. Cl. 600/458; 600/437

(57) ABSTRACT

Ultrasonic diagnostic arrangements (apparatus, methods, etc.) including: an ultrasonic probe or operation for transmitting an ultrasonic wave to an object to be tested and receiving an ultrasonic wave from the object; a transmitter or operation for pulse-driving the ultrasonic probe to transmit an ultrasonic beam to the object; a reception phasing unit or operation for performing phasing on reflected echo signals received by the ultrasonic probe, the reception phasing unit separately performing phasing at multiple phasing frequencies on the reflected echo signals received in response to at least one transmission of the ultrasonic beam; an image generator or operation for generating an ultrasonic image based on the phased received signal.

### TECHNICAL FIELD

The present invention relates to an ultrasonic diagnostic apparatus and an ultrasonic contrast imaging method, and, for example, to a technique for receiving and processing reflected echo signals obtained from an area in an object to be tested in which an ultrasonic contrast agent exists.

### BACKGROUND ART

An ultrasonic diagnostic apparatus pulse-drives an ultrasonic vibrator included in an ultrasonic probe to emit an ultrasonic beam to an object to be tested. Also, the ultrasonic diagnostic apparatus receives reflected echo signals generated due to difference in acoustic impedance in a tissue of the object, performs processing such as phasing addition processing, and generates an ultrasonic image to display on a monitor.

It is generally known that frequency components of an ultrasonic pulse include a certain spread of bandwidth as well as the frequency component of the fundamental wave. This spread of frequency distribution tends to be noticeable particularly when using a contrast echo method using an ultrasonic contrast agent.

The contrast echo method is a method of forming an image for diagnosis, such as blood flow diagnosis, affected area identification and the like, using a signal obtained from an ultrasonic contrast agent including microbubbles with a particle diameter of a few micrometers injected into a blood vessel of the object. For example, as described in Patent Document 1, one known method is to irradiate an ultrasonic pulse having a predetermined frequency spectrum and image a nonlinear component of an ultrasonic echo from microbubbles as a contrast agent.

By the way, for each type of diagnosis using microbubbles as a contrast agent, the same type of microbubbles are used, but their individual particle diameters are not necessarily the same among the microbubbles and are distributed to some extent. It is generally known that, as described in Non-patent Document 1, different particle diameters cause different resonance frequencies.

Accordingly, the frequency distribution of reflected echo signals obtained from an area in which the contrast agent exists particularly tends to be wide and smooth. When such reflected echo signals are phased at a certain phasing frequency, a portion of the bandwidth of the reflected echo signals far from the phasing frequency will have difficulty in contributing to imaging. In other words, only a portion of the microbubbles of the ultrasonic contrast agent in the object may contribute to a focused imaging.

Regarding this point, for example, Patent Document 2 suggests that, in order to extract resonance frequencies from microbubbles having different particle diameters, transmitted signals having different frequency spectrums from one another are transmitted in multiple batches to image ultrasonic echoes from more microbubbles having different particle radiuses.

### PRIOR ART DOCUMENT

### Patent Document

Patent Document 1: JP-A-08-182680

Patent Document 2: JP-A-2007-222610

### Non-Patent Document

Non-patent Document 1: N. de Jong, F. J. Ten Cate et al., “Principles and recent developments in ultrasound contrast agents,” Ultrasonics, 1991, Vol 29, July

### SUMMARY OF THE INVENTION

### Problems to be Solved by the Invention

The method described in Patent Document 2 intends to cause more microbubbles of the contrast agent to contribute to imaging. However, this method is undesirable in that it needs multiple ultrasonic transmission to and reception from the object, which leads to lowering the frame rate.

In view of the above, it is an object of the present invention to provide an ultrasonic diagnostic apparatus and an ultrasonic contrast imaging method that can improve the image quality of an ultrasonic image by effectively utilizing frequency components included in reflected echo signals, while reducing the lowering of the frame rate.

### Means for Solving the Problems

In order to achieve the above object, an ultrasonic diagnostic apparatus in accordance with the invention is characterized by including: an ultrasonic probe for transmitting an ultrasonic wave to an object to be tested and receiving an ultrasonic wave from the object; a transmitter for pulse-driving the ultrasonic probe to transmit an ultrasonic beam to the object; a reception phasing unit for performing phasing on reflected echo signals received by the ultrasonic probe, the reception phasing unit separately performing phasing at multiple phasing frequencies on the reflected echo signals received in response to at least one transmission of the ultrasonic beam; an image generator for generating an ultrasonic image based on the phased received signal; and a display for displaying the generated ultrasonic image.

According to this, even when the frequency distribution of reflected echo signals has a spread, the reflected echo signals are separately phased at multiple phasing frequencies appropriately selected according to the reflected echo signals, which allows frequency components included in the reflected echo signals to be effectively utilized to improve the image quality of an ultrasonic image. Also, what is needed is only phasing at the multiple phasing frequencies the reflected echo signals received in response to, for example, one transmission of the ultrasonic beam, and multiple ultrasonic transmission and reception is not necessary, which can reduce the lowering of the frame rate.

Further, an ultrasonic contrast imaging method in accordance with the invention is characterized by including: a first step in which a transmitter pulse-drives an ultrasonic probe to transmit an ultrasonic beam to the object; a second step in which the ultrasonic probe receives reflected echo signals from the object resulting from transmitting the ultrasonic beam to the object; a third step in which a reception phasing unit performs phasing on the reflected echo signals, the reception phasing unit separately performing phasing on the reflected echo signals received in response to at least one transmission of the ultrasonic beam, at multiple phasing frequencies from an area in which an ultrasonic contrast agent injected into the object exists; and a fourth step in which an image generator generates an ultrasonic image based on the phased received signal.

Particularly, such a phasing is preferably performed on the reflected echo signals from an area in which the ultrasonic contrast agent injected into the object exists. The frequency distribution of the reflected echo signals from an area in which the contrast agents exists tends to spread noticeably. However, the reflected echo signals can be phased separately at the multiple phasing frequencies to be imaged, which allows the entire frequency bands of the reflected echo signals to contribute to imaging. In other words, information can be obtained from more microbubbles of the ultrasonic contrast agent simultaneously, which allows the entire microbubbles to contribute to imaging, providing more sensitively recognizable contrast imaging using microbubbles.

Further, the ultrasonic contrast agent may be a mixture of multiple types of ultrasonic contrast agents. Thus, information from microbubbles having different characteristics can be obtained simultaneously, and a stable contrast image can be obtained in more time phases. Also, this enables image forming according to a purpose of contrast imaging using the contrast agent within the object.

For example, in order to achieve sufficient contrast enhancement using a contrast agent on a minute area such as peripheral blood vessel, the contrast agent desirably has a smaller particle diameter. On the other hand, since it takes time for the contrast agent to reach the peripheral area, the contrast agent desirably has a more stable structure in order to exist in the blood for a long time. Thus, a mixture of multiple types of ultrasonic contrast agents, such as a contrast agent having a small particle diameter and a contrast agent having a stable structure, allows the microbubbles to travel into the peripheral area without being damaged, while improving the image quality of an ultrasonic image of the peripheral area. Note that appropriately changing the mixture ratio of the multiple types of contrast agents allows the selective highlighting of a contrast image of a specific area in a specific time phase.

Further, when using the multiple types of ultrasonic contrast agents, the reception phasing unit can include as the multiple phasing frequencies at least one of difference and sum of frequencies from different resonance frequencies included in reflected echo signals obtained from an area in the object in which the multiple types of ultrasonic contrast agents exist.

Further, displaying in time series on the display the frequency distribution of reflected echo signals obtained from an area in the object in which the multiple types of ultrasonic contrast agents exist can provide a user with information useful for diagnosis. For example, displaying the movement of the frequency distribution of reflected echo signals can help the user recognize how the multiple types of contrast agents are flowing into an area of interest, or in what time phase a desired contrast agent flows into an area of interest.

Further, the reception phasing unit can be configured so that multiple phasing frequencies are selected based on the frequency distribution of reflected echo signals obtained from an area in the object in which the multiple types of ultrasonic contrast agents exist. According to this, even when the frequency distribution of reflected echo signals moves with time phase due to the multiple types of contrast agents, for example, detecting a peak frequency from the frequency distribution of reflected echo signals in each time phase and using the peak frequency as phasing frequency allows phasing to be performed at an optimum phasing frequency in every time phase.

### Advantage of the Invention

According to the invention, an ultrasonic diagnostic apparatus and an ultrasonic contrast imaging method can be provided that can improve the image quality of an ultrasonic image by effectively utilizing frequency components included in reflected echo signals, while reducing the lowering of the frame rate.

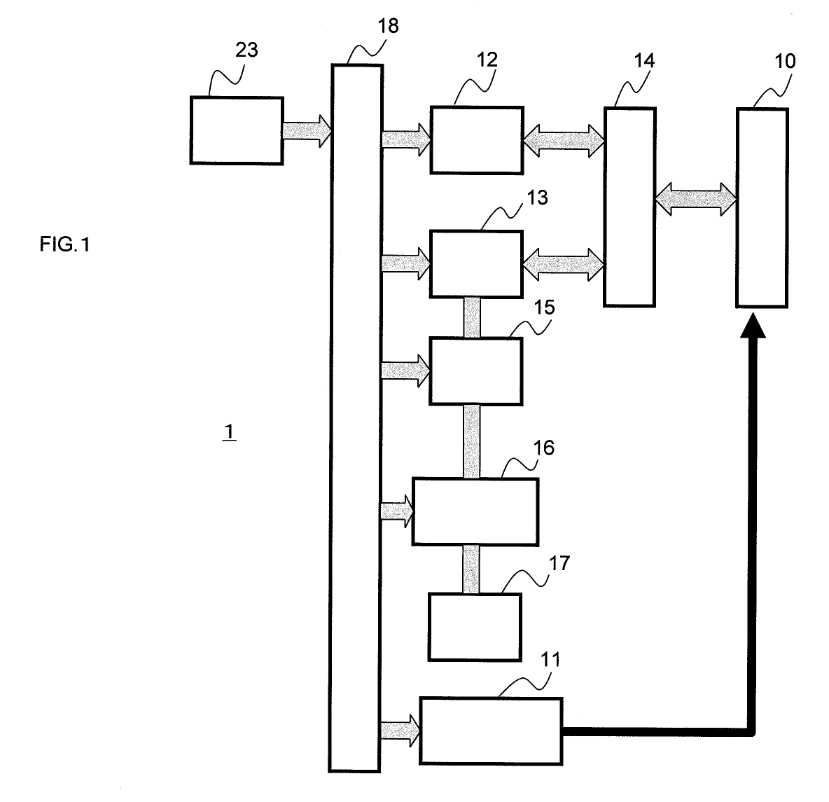

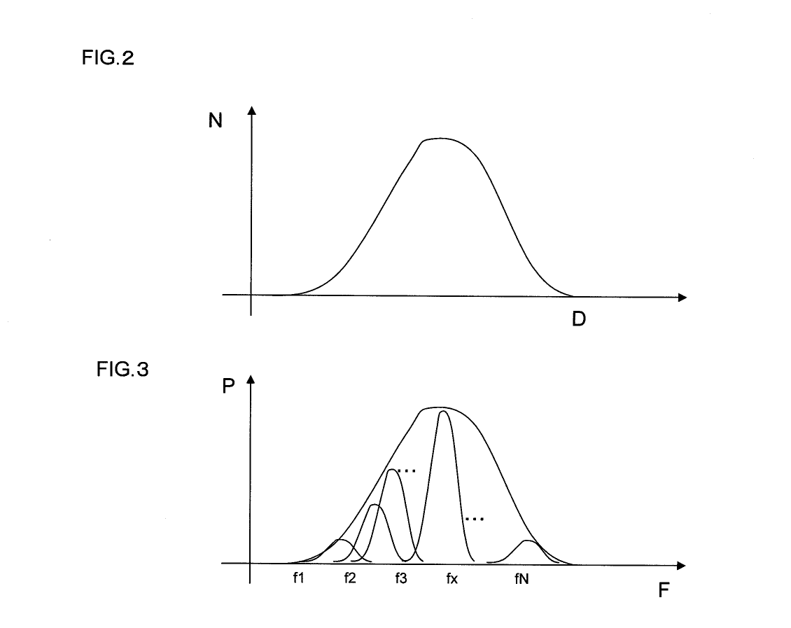

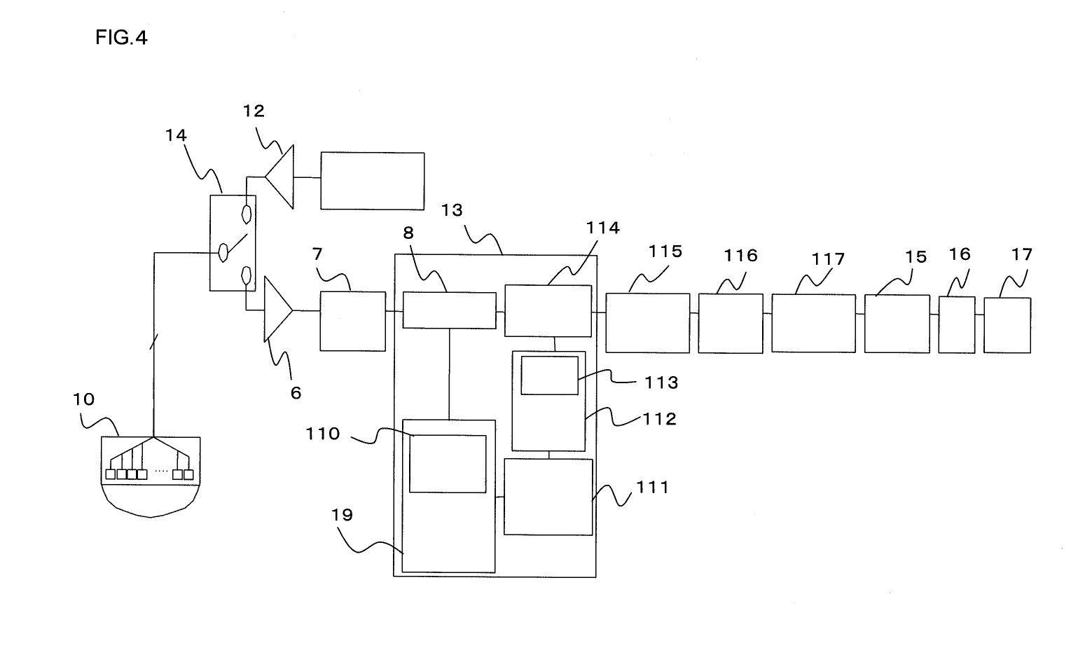

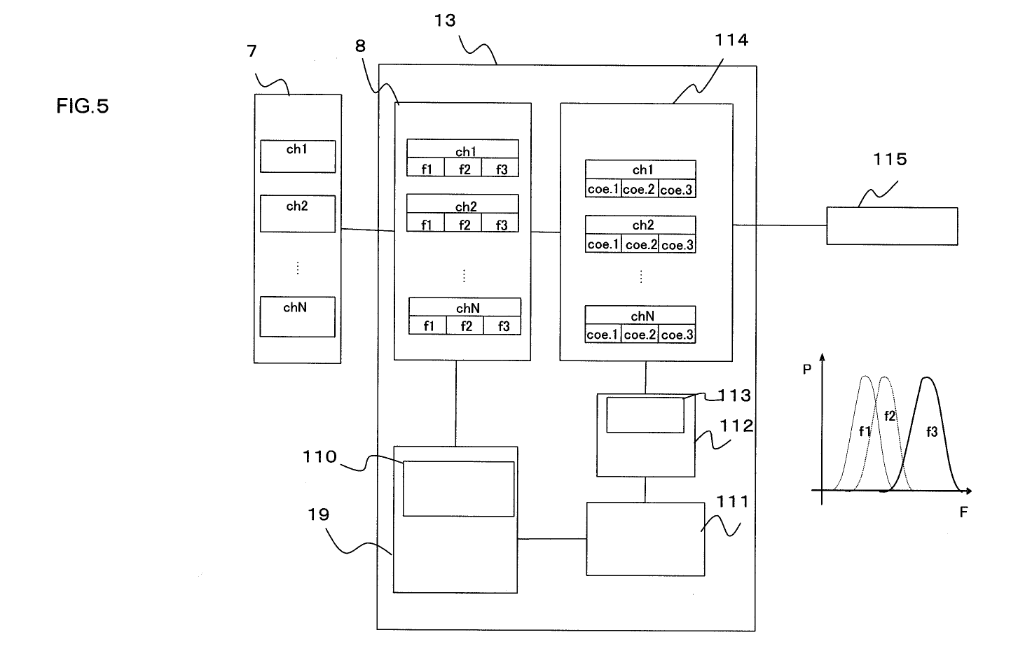

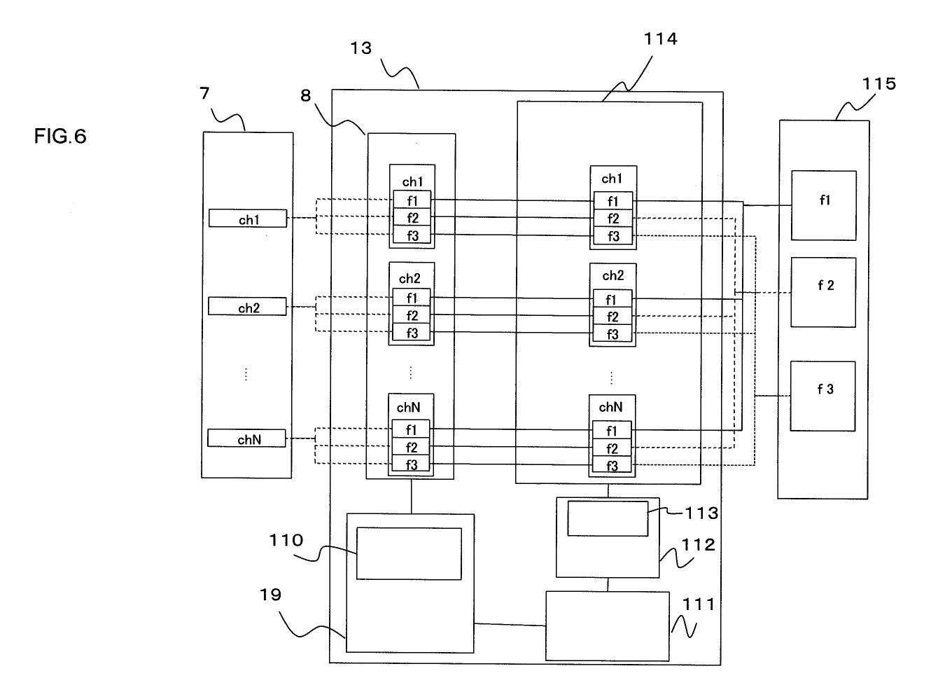

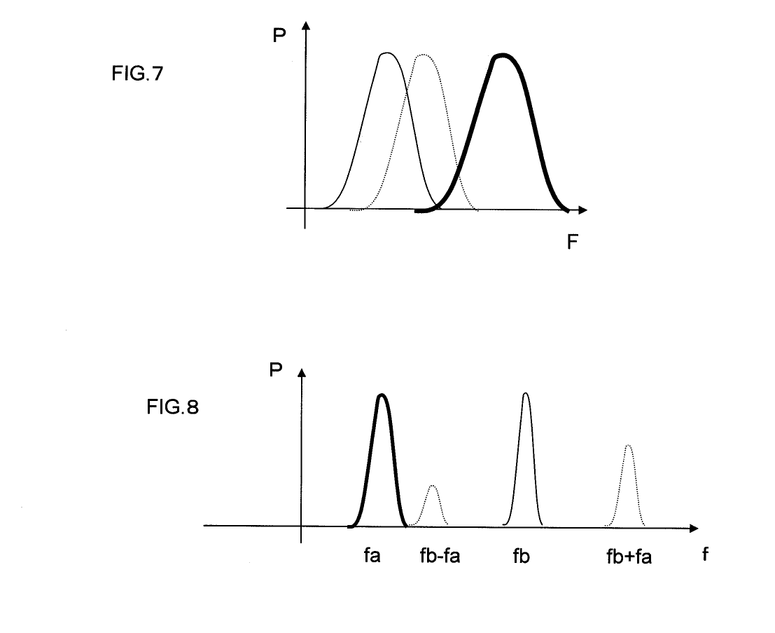

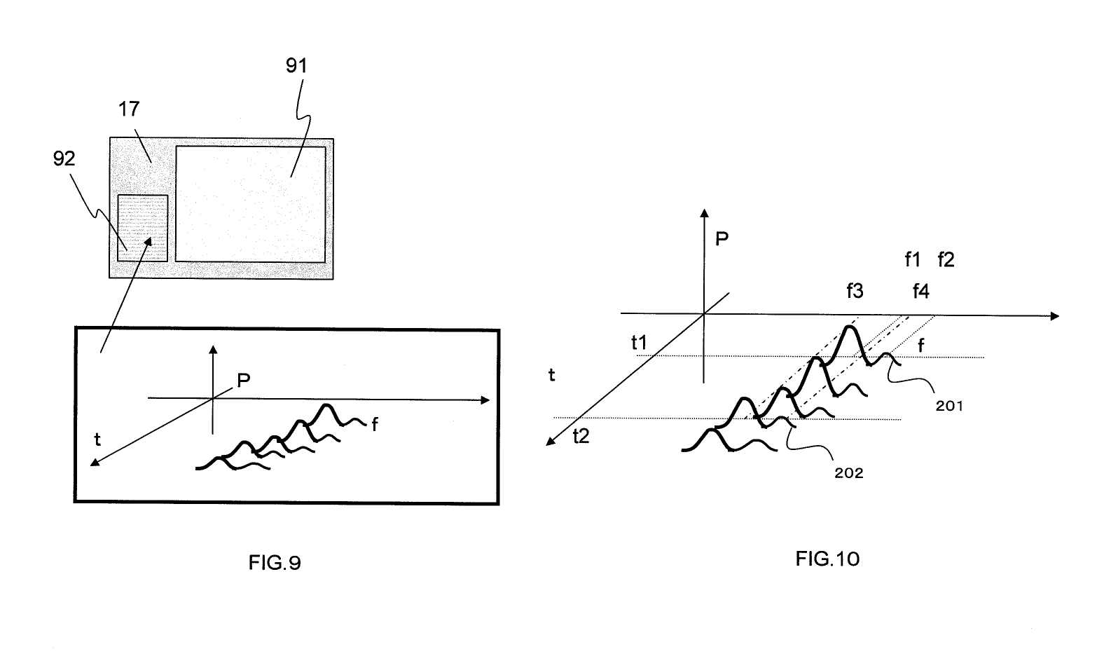

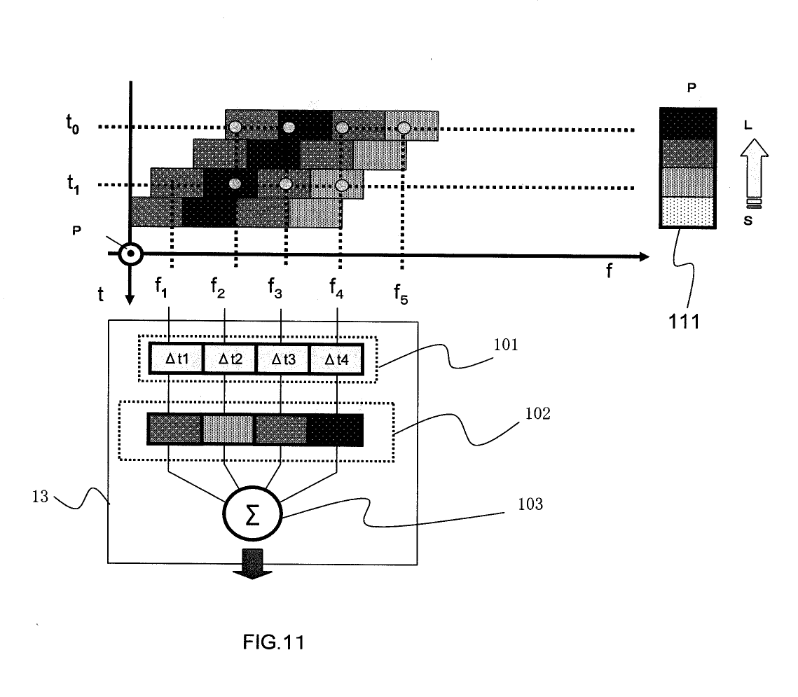

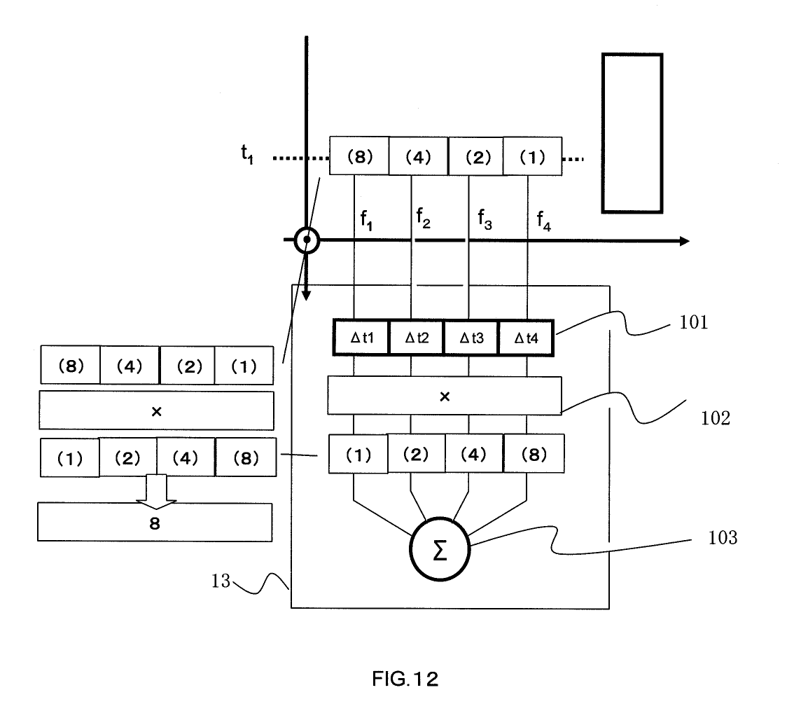

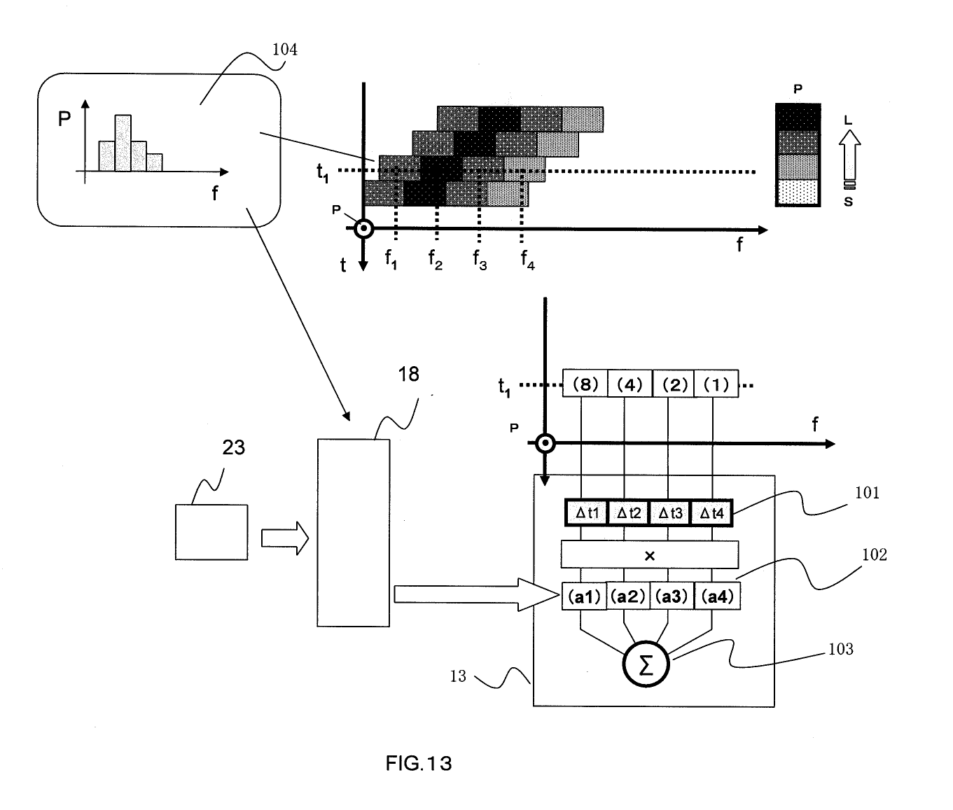

### BRIEF DESCRIPTION OF THE DRAWINGS

[FIG. 1] A block diagram showing an entire configuration of an ultrasonic diagnostic apparatus in accordance with a first embodiment.

[FIG. 2] A graph schematically showing the particle diameter distribution of an ultrasonic contrast agent.

[FIG. 3] A graph schematically showing the resonance frequency distribution of the ultrasonic contrast agent.

[FIG. 4] A diagram showing a detailed configuration of a reception phasing unit and pre- and post-processing function blocks of the reception phasing unit not shown in FIG. 1.

[FIG. 5] A diagram showing a concept of phasing in time-division manner in the reception phasing unit.

[FIG. 6] A diagram showing a concept of phasing in parallel manner in the reception phasing unit.

[FIG. 7] A graph schematically showing the resonance frequency distribution when the ultrasonic contrast agent includes materials having different outer shells.

[FIG. 8] A graph showing that reflected echo signals from an area in an object into which two types of contrast agents having different resonance frequencies are injected indicate the frequencies Fa and Fb.

[FIG. 9] A diagram showing an example of displaying a diagnostic image and the frequency distribution of reflected echo signals in time series on a monitor.

[FIG. 10] An graph showing a concept of the relation between the frequency distribution of reflected echo signals in each time phase and the selected phasing frequencies.

[FIG. 11] A diagram showing a concept of adjusting a signal level of a reflected echo signal for each band in the reception phasing unit.

[FIG. 12] A diagram showing a further detail of the signal level adjusting capability of the reception phasing unit.

[FIG. 13] A diagram showing a specific example of configuration to implement the signal level adjusting capability of the reception phasing unit.

### MODE FOR CARRYING OUT THE INVENTION

Embodiments of an ultrasonic diagnostic apparatus in accordance with the invention are described below. In the description below, like functional components are denoted by like numerals, and will not be repeatedly described.

### First Embodiment

FIG. 1 is a block diagram showing an entire configuration of the ultrasonic diagnostic apparatus in accordance with a first embodiment. An ultrasonic diagnostic apparatus 1 includes: an ultrasonic probe 10 including multiple vibrators; an element selector 11 for selecting an element of the vibrators; a transmitter 12 for transmitting a signal to the ultrasonic probe 10; a reception phasing unit 13 for phasing a signal received from the ultrasonic probe 10; and a transmission/reception separator 14 for switching between the transmitter 12 and the reception phasing unit 13.

Further, the ultrasonic diagnostic apparatus 1 includes: a signal processor 15 for processing a signal from the reception phasing unit 13; a scan converter 16 for scan-converting from ultrasonic scanning to display scanning using a signal from the signal processor 15; a monitor 17, including a CRT, liquid crystal display or the like, for displaying an image data from the scan converter 16; a controller 18 for controlling various components; and an input section 23 for inputting a control signal to the controller 18.

The transmitter 12 provides a drive signal to an ultrasonic vibrator to transmit an ultrasonic beam into an object to be tested. The transmitter 12 includes a known pulse generator circuit, a known amplifier circuit and a known delay circuit for transmission.

The reception phasing unit 13 phases reflected echo signals that are electric signals (received signals) converted by the ultrasonic vibrator from an ultrasonic wave transmitted into the object and reflected from within the object. The reception phasing unit 13 includes a known delay circuit and the like. The transmission/reception separator 14 switches the signal direction depending on whether transmission or reception is occurring.

The signal processor 15 performs logarithmic conversion, filtering, γ correction and the like as preprocessing for imaging a received signal output from the reception phasing unit 13.

The scan converter 16 accumulates a signal output from the signal processor 15 for each ultrasonic beam scanning to form an image data and outputs the image data according to the scanning of an image display device, that is, performs scan conversion from ultrasonic scanning to display scanning.

The monitor 17 is a display device for displaying as an image an image data (converted to a luminance signal) output from the scan converter 16.

The controller 18 is a central processing unit (CPU) for directly or indirectly controlling the above-described components to perform ultrasonic transmission/reception and image displaying.

Next, an operation of the ultrasonic diagnostic apparatus is described. The ultrasonic probe 10 is touched to an area to be tested of the object. A scan parameter such as transmission focus depth is input from the input section 23. Then, an instruction to start ultrasonic scanning is input. The controller 18 controls the units to start ultrasonic scanning. First, the controller 18 outputs to the element selector 11 and the transmitter 12 an instruction to select a vibrator to be used in the first transmission, an instruction to output a drive pulse and an instruction to set a delay time according to the transmission focus depth.

When these instructions are executed, the transmitter 12 provides the drive pulse to the ultrasonic probe 10 via a transmission delay circuit (not shown). A vibrator in the ultrasonic probe 10 determined by the element selector 11 and the transmitter 12 that provides a transmitted signal are connected via the transmission/reception separator 14. When the drive pulse is input, the vibrators vibrate at predetermined frequencies and sequentially transmit an ultrasonic wave into the object.

When the ultrasonic wave is transmitted into the object, a portion of the wave is reflected by a surface of a tissue or organ in a living body at which acoustic impedance changes, toward the ultrasonic probe 10 as reflected echoes. The controller 18 controls the reception system to receive the reflected echoes. Specifically, first, upon finishing the transmission, the element selector 11 performs switching selection to connect a vibrator for reception with the reception phasing unit.

With this vibrator switching selection, control of reception delay time is performed on the reception phasing unit 13.

Received signals output from reception delay circuits are phased and subjected to various processings (described later) by the reception phasing unit 13, and then output to the signal processor 15 as a received beam signal. The signal processor 15 performs the above-described processing on the input received signal and outputs the processed signal to the scan converter 16. The scan converter 16 stores the input signal in a memory (not shown) and reads to output the stored contents to the monitor 17 according to a synchronization signal for displaying.

Upon finishing the above operation, the controller 18 changes the direction of ultrasonic transmission/reception to perform the second round of the operation, and then performs the third round and so on. In this way, the controller 18 sequentially changes the direction of ultrasonic transmission/reception to repeat the above operation.

Next, the operation of a contrast echo method for using microbubbles to obtain a contrast image is described. First, an ultrasonic contrast agent provided in powder form is suspended in an injection solvent just before using. Then, the suspension is injected into a vein. The contrast agent travels through the vein to the heart and then the lungs, then returns from the lungs to the heart through an artery, and then circulates throughout the body.

On the way of circulation, the contrast agent is excited by an ultrasonic wave that is generated by applying to the ultrasonic probe 10 an impulse-like waveform, having various frequency components, transmitted from the transmitter 12. In response to the transmitted signal having such a wide frequency bandwidth, though limited by the frequency bandwidth of the ultrasonic probe 10, the microbubbles of the injected contrast agent perform expiratory movement at their own resonance frequencies to emit their-own-frequency signals.

That is, the contrast agent emits not only a signal of the transmission frequency Ft but also signals of a constant multiple of Ft and signals of Ft divided by a constant due to a nonlinear contraction referred to as expiratory movement. Among others, a signal of twice Ft is emitted relatively strongly, so the twice Ft component is used to image an area in which the contrast agent is concentrated.

According to such a contrast echo method, to cite contrast imaging of the liver as an example, a malignancy of the liver takes nutrition from an artery, so the contrast agent flowing through the artery to the liver is concentrated at the malignancy, allowing the ultrasonic diagnostic apparatus to display the malignancy brightly.

On the other hand, blood having reached the intestines and taken nutrition then travels through the portal vein to reach the liver and is supplied to a healthy liver tissue. As a result, in diagnosis of the liver, first, the malignancy is contrast-imaged, then the entire liver tissue is displayed.

By the way, the resonance frequency due to the expiratory movement of the contrast agent applied with the ultrasonic wave is expressed by Eq. 1, as described in Non-patent Document 1, for example.

$${F}_{T} = {\frac{1}{2 π R} [ \frac{3 YP}{ρ} ]}^{\frac{1}{2}} \qquad [ Eq . 1 ]$$

FT: resonance frequency, R: microbubble radius, y: heat capacity, P: pressure, p: density of medium around microbubble

As seen from this equation, the resonance frequency of the contrast agent depends on the microbubble size, and the pressure to the microbubble. Although the particle diameter distribution of generally used contrast agent is within a certain range, it is still thought that the maximum radius is nearly twice larger than the minimum radius. Thus, in general, the particle diameter distribution of the contrast agent is as shown in FIG. 2. In the graph of FIG. 2 showing the particle diameter distribution, the horizontal axis indicates the particle diameter (D), and the vertical axis indicates the number (N).

As described above, reflected echo signals having resonance frequencies ranging by a factor of two are emitted from the various microbubbles, as shown in FIG. 3. In the graph of FIG. 3 showing the frequency distribution, the horizontal axis indicates the resonance frequency (F), and the vertical axis indicates the power (P) of reflected echo signals. For example, the reflected echo signals have a frequency distribution ranging from F1 to FN.

When the reflected echo signals having such a spread of frequency distribution are phased at a certain phasing frequency in a conventional way, a portion of the bandwidth of the reflected echo signals far from the phasing frequency will have difficulty in contributing to imaging. In other words, only a portion of the microbubbles of the ultrasonic contrast agent in the object may contribute to the imaging.

Next, the reception phasing unit 13 is described, which is a feature of the ultrasonic diagnostic apparatus of the embodiment to address the above problem. FIG. 4 shows a detailed configuration of the reception phasing unit 13 and pre- and post-processing function blocks of the reception phasing unit 13 not shown in FIG. 1.

As shown in FIG. 4, the reflected echo signals obtained from the ultrasonic probe 10 through the transmission/reception separator 14 are amplified by a reception amplifier 6. Then, an AID converter 7 digitizes the amplified signals, then time-divides the digitized signals by the number of bands required for frequency compounding, and then outputs the time-divided signals to the reception phasing unit 13. In general, the frequency compounding is a technique for improving the uniformity of lateral resolution and resolution for an area of interest by separately signal-processing low- and high-frequency components and adding those components. In the reception phasing unit 13, a different center phasing frequency is used for each time division timing. A condition for maximizing the spatial resolution is unique to each band. Parameters for determining the condition include an aperture width, amplitude weighting coefficients for element channels forming the aperture and the like.

Specifically, as shown in FIG. 4, the reception phasing unit 13 includes: a center phasing frequency setting section 111 for setting a reception phasing frequency; a focus data calculation section 19 using this frequency to calculate a focus data; a focus data storing memory 110 for storing the calculated data; and a delay amount correction section 8 using the stored data to perform reception phasing. Note that, instead of using the focus data calculation section 19, the focus data may be externally calculated and transferred to the focus data storing memory 110.

Multiple center phasing frequencies to be used for forming an image to be subjected to frequency compounding are input from the controller 18. Then, conditions for maximizing the spatial resolution for each of the frequencies are calculated by the controller 18. These conditions may also be given in a table in advance in a storage medium such as a memory. The reception phasing unit 13 uses these condition to perform amplitude weighting and aperture width determination and performs high-spatial resolution phasing using a separate condition for each different center frequency.

Also, the reception phasing unit 13 includes: a beam forming condition calculation section 112 for calculating a condition for forming an optimum reception beam; a storing memory 113 for storing the calculated forming condition; and a beam forming processing section 114 for calculating an optimum focus data based on the stored data. A different weighting coefficient is given to a channel data for each vibrator. Any change can be made to this coefficient. This coefficient can be changed so as to increase the weight of the image data phased to have the highest spatial resolution.

The signals of the vibrators of the ultrasonic probe 10 output from the beam forming processing section 114 are added by a channel adder 115. Then, a band divider 116 divides the added signal into a center frequency-band signal and the remaining frequency-band signal. The band signal adder 117 adds the center frequency-band signal and the remaining frequency-band signal.

The multiple-band processing has been described with reference to, but is not limited to, the time-division processing. A parallel processing may also be possible by giving a separate circuit to a processing system for each center phasing frequency.

Also, for the phasing condition selected for each frequency, the spatial resolution for each band is not necessarily given priority because it is the most important that the diagnostic image after the addition is easy for a diagnostician to read.

FIGS. 5 and 6 show concepts of performing time-division processing and parallel processing, respectively, in the reception phasing unit 13. For example, in FIGS. 5 and 6, phasing is performed at three phasing frequencies F1 to F3.

As shown in FIG. 5, in performing time-division processing, a received data for each vibrator channel of the ultrasonic probe 10 is sequentially delayed by a separate amount of delay for each of the phasing frequencies F1 to F3 in this order in the delay amount correction section 8, then is subjected to amplitude weighting and aperture width control in the beam forming processing section 114. In the graph in the lower right of FIG. 5 showing the frequency distribution, the horizontal axis indicates the resonance frequency (F), and the vertical axis indicates the power (P) of reflected echo signals.

On the other hand, as shown in FIG. 6, in performing parallel processing, a received data for each vibrator channel is delayed by a separate amount of delay for each of the phasing frequencies F1 to F3 in their respective separate lines, then is subjected to amplitude weighting and aperture width control in the beam forming processing section 114 in the same separate lines.

As described above, according to this embodiment, the reflected echo signals from the area in which the contrast agent exists are phased separately at the multiple phasing frequencies to be imaged, which allows the entire frequency bands of the reflected echo signals to contribute to imaging. In other words, information can be obtained from more microbubbles of the ultrasonic contrast agent simultaneously, which allows the entire microbubbles to contribute to imaging, providing more sensitively recognizable contrast image using microbubbles. Also, in this embodiment, what is needed is only phasing at the multiple phasing frequencies the reflected echo signals received in response to one transmission of the ultrasonic beam, and multiple ultrasonic transmission and reception is not necessary, which can reduce the lowering of the frame rate.

However, if the frame rate required for imaging has sufficient room, two or more transmissions may be allowed. The more the number of measurements (transmissions) is, the higher the accuracy of the measurement data can be. One transmission or multiple transmissions may also be switchable so that it can be selected whether priority is to be given to the frame rate or the accuracy.

Phasing according to this embodiment is suitable for the reflected echo signals from the area in which the contrast agent exists, but is not limited to this. In general, frequency components of an ultrasonic pulse have a certain spread of bandwidth as well as the frequency component of the fundamental wave. The above-described phasing is also applicable to reflected echo signals having such a spread of frequency band.

According to this, even when the frequency distribution of reflected echo signals has a certain spread, the reflected echo signals are separately phased at multiple phasing frequencies suited to the reflected echo signals, which allows frequency components included in the reflected echo signals to be effectively utilized to improve the image quality of an ultrasonic image.

### Second Embodiment

Next, a second embodiment of the ultrasonic diagnostic apparatus in accordance with the invention is described. This embodiment is different from the first embodiment only in that a mixture of multiple types of ultrasonic contrast agents is used as an ultrasonic contrast agent to be injected into the object. So, the remaining portion similar to that of the first embodiment is not repeatedly described.

As seen from Eq. 1 above, the resonance frequency of an ultrasonic contrast agent also depends on pressure. This means the resonance frequency depends on the sound pressure of ultrasonic transmission and the hardness of the outer shell forming microbubbles. In other words, the behavior of microbubbles varies depending on sound pressure or mechanical index (MI) or the hardness of the outer shell of the microbubbles.

Accordingly, when an ultrasonic contrast agent includes materials having different outer shells, the resonance frequency is distributed, for example, as shown in FIG. 7. In the graph of FIG. 7 showing the frequency distribution, the horizontal axis indicates the resonance frequency (F), and the vertical axis indicates the power (P) of reflected echo signals. In this case, as shown in FIG. 7, since three peak resonance frequencies exist, in reception phasing, phasing at the three peak frequencies as phasing frequency allows a received image to be sensitively constructed for each of the peak frequencies.

In addition, this embodiment intends to achieve uniform contrast enhancement under various conditions by selectively use contrast agents to be imaged. For example, contrast agents including different materials forming outer shells are used simultaneously as contrast agent to be used as described above, which enables image forming according to a purpose of contrast imaging using the contrast agent within the body.

For example, in order to achieve sufficient contrast enhancement using a contrast agent on a minute area such as peripheral blood vessel, the contrast agent desirably has a smaller particle diameter. Further, since it takes time for the contrast agent to reach the peripheral area, the contrast agent desirably has a more stable structure in order to exist in the blood for a longtime. Accordingly, in contrast-imaging the minute area such as peripheral area, for example, the contrast agent that is more stable in liquid and has a small particle diameter is used to form an image. In contrast-imaging the other area, a contrast agent that has a lower resonance frequency is used to form an image.

This allows the microbubbles to travel into the peripheral area without being damaged. Then, adding contrast images obtained from those areas can provide a contrast image achieving more effective contrast enhancement than before. Also, for an area such as cancer in which relatively thin vessels are gathering, contrast imaging is performed using microbubbles having high resonance frequencies, enabling contrast image forming focusing the cancer, for example.

Also, areas into which contrast agents having different characteristics flow can be selectively contrast-imaged by selecting a different resonance frequency depending on the diameter of microbubbles (contrast agent) and the like.

In general, contrast agents having small particle diameters easily flow into a minute area. Based on this, imaging using high resonance frequencies is described in the above example. However, the embodiment is not limited to this.

Also, in using the ultrasonic diagnostic apparatus, ultrasonic contrast agents having different characteristics are preferably used with a mixture ratio for each sequence to be used in imaging. For example, when the amount of contrast agents flowing into a peripheral area maybe smaller than that for the other area, larger amount of contrast agents having small particle diameters can be given to maintain the uniform sensitivity of the contrast agents in all the areas.

### Third Embodiment

Next, a third embodiment of the ultrasonic diagnostic apparatus in accordance with the invention is described. This embodiment is different from the first embodiment only in that a mixture of multiple types of ultrasonic contrast agents is used as an ultrasonic contrast agent to be injected into the object, and that difference and sum of frequencies from different resonance frequencies included in reflected echo signals obtained from an area in which the ultrasonic contrast agents exist are included as phasing frequencies. So, the remaining portion similar to that of the first embodiment is not repeatedly described.

In this embodiment, for example, any two frequencies of a signal including multiple resonance frequencies are focused. Then, at least one of the frequency component having the difference of the two frequencies and the frequency component having the sum of the two frequencies is imaged. FIG. 8 shows that reflected echo signals from an area in the object into which two types of contrast agents having different resonance frequencies are injected indicate the frequencies Fa and Fb, for example. In the graph of FIG. 8 showing the frequency distribution, the horizontal axis indicates the resonance frequency (F), and the vertical axis indicates the power (P) of reflected echo signals. In this case, the vibrators observe the sum component Fb+Fa and the difference component Fb−Fa as a result of interference between the sound sources.

Assuming that Fa and Fb are higher-order harmonics of a transmission frequency, Fa and Fb components of a transmission signal is smaller than the fundamental wave component. Also, nonlinearity for a specific frequency of the other portion of tissue than the contrast agents is smaller than that of the contrast agents. So, the ratio of the signal from the other portion of tissue to that from the contrast agents is relatively small at Fb−Fa and Fb+Fa. Thus, the effect of increasing the ratio of the signal from the contrast agents to that from the other portion of tissue is expected.

### Fourth Embodiment

Next, a fourth embodiment of the ultrasonic diagnostic apparatus in accordance with the invention is described. This embodiment is different from the first embodiment only in that a mixture of multiple types of ultrasonic contrast agents is used as an ultrasonic contrast agent to be injected into the object, and that the frequency distribution of reflected echo signals obtained from an area in the object in which the multiple types of ultrasonic contrast agents exist is displayed in time series on the monitor. So, the remaining portion similar to that of the first embodiment is not repeatedly described.

FIG. 9 shows an example of the monitor 17 displaying a diagnostic image 91 and a graph 92 in which the frequency distribution of reflected echo signals is displayed in time series. The graph 92 of the frequency distribution shown in FIG. 9 has three axes at right angles to one another, indicating the time (t), the resonance frequency (F) and the power (P) of reflected echo signals. Displaying the frequency distribution of reflected echo signals in this way can provide a user with information useful for diagnosis. For example, displaying the movement of the frequency distribution of reflected echo signals can help the user recognize how the multiple types of contrast agents are flowing into an area of interest, or in what time phase a desired contrast agent flows into an area of interest.

Although the frequency distribution of reflected echo signals in FIG. 9 is shown in a three-dimensional plot using a three-dimensional axis of the time (t), the frequency (F) and the power (P), but is not limited to this. For example, the frequency distribution may be shown in a two-dimensional plot of the time and the frequency with the frequency intensity displayed color-coded, or in a line graph of a few peak values in descending order of the power (P).

### Fifth Embodiment

Next, a fifth embodiment of the ultrasonic diagnostic apparatus in accordance with the invention is described. This embodiment is different from the first embodiment only in that a mixture of multiple types of ultrasonic contrast agents is used as an ultrasonic contrast agent to be injected into the object, and that multiple phasing frequencies are selected based on the frequency distribution of reflected echo signals obtained from an area in the object in which the multiple types of ultrasonic contrast agents exist. So, the remaining portion similar to that of the first embodiment is not repeatedly described.

FIG. 10 shows a concept of the relation between the frequency distribution of reflected echo signals in each time phase and the selected phasing frequencies. The graph of the frequency distribution shown in FIG. 10 has three axes at right angles to one another, indicating the time (t), the resonance frequency (F) and the power (P) of reflected echo signals. For example, it is assumed that, when t1 elapses from the injection of the contrast agent, a frequency distribution 201 of reflected echo signals from an area of interest is given. The frequency distribution 201 has two peak frequencies F1 and F2. When F1 and F2 are set as a frequency of interest, the reception phasing unit 13 performs phasing at these frequencies.

Also, it is assumed that, when t2 elapses, a frequency distribution 202 of received signals from the area of interest is given. In this case, the frequency distribution 202 has two peak frequencies F3 and F4. When F3 and F4 are set as a frequency of interest, the reception phasing unit 13 performs phasing at F3 and F4 at t2.

According to this embodiment, reception at a frequency at which the signal from the contrast agent is at maximum intensity is possible in every time phase. Although, in FIG. 10, two frequencies in descending order of the power are selected to perform phasing, but this embodiment is not limited to this, and phasing frequencies can be selected as appropriate.

### Sixth Embodiment

Next, a sixth embodiment of the ultrasonic diagnostic apparatus in accordance with the invention is described. This embodiment is different from the first embodiment in that a mixture of multiple types of ultrasonic contrast agents is used as an ultrasonic contrast agent to be injected into the object, and that the reception phasing unit is provided with a capability of controlling a signal level so that a reflected echo signal intensity for each band is equalized, and the like. So, the remaining portion similar to that of the first embodiment is not repeatedly described.

FIG. 11 shows a concept of adjusting a signal level of a reflected echo signal for each band in the reception phasing unit 13. The upper block diagram shows the intensity of a reflected echo signal from the contrast agent for each time and frequency. The horizontal axis indicates the frequency, the vertical axis indicates the time, and the axis perpendicular to the page indicates the signal intensity. Thus, the upper diagram shows how the frequency distribution of the intensity of a reflected echo signal from the contrast agent changes along with time.

For example, according to the diagram, at time to, a received signal from the contrast agent exists in the frequency range from F2 to F5. At time t1, the received signal exists in the frequency range from F1 to F4, and F5 component that existed at time t0 does not exist.

Now, it is assumed that, as shown in FIG. 11, in the frequency distribution of the reflection intensity of the signal at time t1, the power P of F2 is the largest, the power P of F1 and F3 are the second largest and the same, and the power P of F4 is the smallest. In order to image this, the reception phasing unit 13 delays each channel signal for each frequency band in a delay unit 101. In delaying, when the received signal intensity is different for each frequency band, adding the received signals of the individual bands as they are results in a received image highly depending on the received signal intensity for each band.

Specifically, in a space in which reflectors having a frequency component with high signal intensity are distributed, the power P is displayed as larger, and in a space in which reflectors having a frequency component only with low signal intensity are distributed, the power P is displayed as smaller, resulting in a patchy image.

In order to avoid this, in performing the addition for individual bands, a weighted multiplier 102 performs weighting based on a power scale 105 shown in the far-right of FIG. 11. In the power scale 111, color phases are arranged in ascending order of power from bottom to top. In this example, if the reflected signal intensity is denoted by P(F), the signal intensity at time t1 can be expressed as P(F2)>P(F1, F3)>P(F4). If the magnitude of weight is denoted by W(F), W(F4)>W(F1, F3)>W(F2) holds. Considering these allows the reflected signal intensity for each band to be corrected. The signals of the individual bands with the reflected signal intensity corrected in this way are added by a band adder 103.

FIG. 12 shows a further detail of the signal level adjusting capability of the reception phasing unit in accordance with this embodiment. It is assumed that the signal intensities of bands F1, F2, F3 and F4 at time t1 are “8,” “4,” “2” and “1,” respectively. If these intensities are added as they are, spatial distributions of signals in those bands are imaged, resulting in a highly patchy image.

In order to avoid such a patchy image and display with a uniform intensity the area in which the contrast agent itself exists, weights of “1,” “2,” “4” and “8” are given to F1, F2, F3 and F4, respectively, so as to cancel the difference in the signal intensities. Consequently, all the signal intensities of the individual bands become “8” to form an image having a spatially-uniform intensity.

FIG. 13 shows a specific example of configuration to implement the signal level adjusting capability of the reception phasing unit in accordance with this embodiment. P(F) (denoted by reference numeral 104 in FIG. 9), the value of signal intensity read for each band as shown in FIG. 9, is sent to the controller 18 at regular time intervals. To display with the same signal intensity for each band, the controller 18 only needs to calculate the reciprocal 1/P(F) and give a value proportional to 1/P(F) as a weight to the weighted multiplier 102.

This embodiment have been described with reference to equalizing the intensities of individual frequency bands, but is not limited to this. To highlight only a specific resonance frequency component, a weighting value may be given only to that frequency component and zero or very small value may be given to the other components.

In this case, a weighting function is manually input to the input section 23 and sent through the controller 18 to the weighted multiplier in the reception phasing unit 13 in which multiplication is performed.

As described above, the weighting function is not uniquely determined, but may take any value. Further, the weighting may be the addition for each band rather than the processing in the reception phasing unit.

### DESCRIPTION OF REFERENCE NUMERALS AND SIGNS

1 ultrasonic diagnostic apparatus, 10 ultrasonic probe, 12 transmitter, 13 reception phasing unit, 15 signal processor, 16 scan converter, 17 monitor, 18 controller, 19 focus data calculation section, 23 input section, 102 weighted multiplier, 110 focus data storing memory, 111 center phasing frequency setting section, 112 beam forming condition calculation section, 114 beam forming processing section, 115 channel adder, 116 band divider, 117 band signal adder

1. An ultrasonic diagnostic apparatus, comprising:
an ultrasonic probe for transmitting an ultrasonic wave to an object to be tested and receiving an ultrasonic wave from the object;
a transmitter for pulse-driving the ultrasonic probe to transmit an ultrasonic beam to the object;
a reception phasing unit for performing phasing on reflected echo signals received by the ultrasonic probe, the reception phasing unit separately performing phasing at multiple phasing frequencies on the reflected echo signals received in response to at least one transmission of the ultrasonic beam;
an image generator for generating an ultrasonic image based on the phased received signal; and
a display for displaying the generated ultrasonic image.

2. The ultrasonic diagnostic apparatus according to claim 1, wherein the reception phasing unit performs the phasing on the reflected echo signals from an area in which an ultrasonic contrast agent injected into the object exists.

3. The ultrasonic diagnostic apparatus according to claim 2, wherein the ultrasonic contrast agent is a mixture of multiple types of ultrasonic contrast agents, and the reception phasing unit performs the phasing on the reflected echo signals from an area in the object in which an ultrasonic contrast agent exists, based on eigen values of the multiple ultrasonic contrast agents.

4. The ultrasonic diagnostic apparatus according to claim 3, wherein the reception phasing unit includes as the multiple phasing frequencies at least one of difference and sum of frequencies from different resonance frequencies included in reflected echo signals obtained from an area in the object in which the multiple types of ultrasonic contrast agents exist.

5. The ultrasonic diagnostic apparatus according to claim 3, further comprising a display controller for controlling to display in time series on the display the frequency distribution of reflected echo signals obtained from an area in the object in which the multiple types of ultrasonic contrast agents exist.

6. The ultrasonic diagnostic apparatus according to claim 3, wherein the reception phasing unit selects the multiple phasing frequencies based on the frequency distribution of reflected echo signals obtained from an area in the object in which the multiple types of ultrasonic contrast agents exist.

7. An ultrasonic contrast imaging method, characterized by comprising:
a first step in which a transmitter pulse-drives an ultrasonic probe to transmit an ultrasonic beam to an object to be examined;
a second step in which the ultrasonic probe receives reflected echo signals from the object in response to transmitting the ultrasonic beam to the object;
a third step in which a reception phasing unit performs phasing on the reflected echo signals, the reception phasing unit separately performing phasing on the reflected echo signals received in response to at least one transmission of the ultrasonic beam, at multiple phasing frequencies from an area in which an ultrasonic contrast agent injected into the object exists; and
a fourth step in which an image generator generates an ultrasonic image based on the phased received signal.

8. The ultrasonic contrast imaging method according to claim 7, wherein the ultrasonic contrast agent is a mixture of multiple types of ultrasonic contrast agents, and, in the fourth step, the reception phasing unit performs the phasing on the reflected echo signals from an area in the object in which an ultrasonic contrast agent exists, based on eigen values of the multiple ultrasonic contrast agents.

9. The ultrasonic contrast imaging method according to claim 8, wherein, in the fourth step, the reception phasing unit includes as the multiple phasing frequencies at least one of difference and sum of frequencies from different resonance frequencies included in reflected echo signals obtained from an area in the object in which the multiple types of ultrasonic contrast agents exist.

10. The ultrasonic contrast imaging method according to claim 8, further comprising a fifth step in which a display controller controls to display in time series on the display the frequency distribution of reflected echo signals obtained from an area in the object in which the multiple types of ultrasonic contrast agents exist.

11. The ultrasonic contrast imaging method according to claim 8, wherein, in the fourth step, the reception phasing unit selects the multiple phasing frequencies based on the frequency distribution of reflected echo signals obtained from an area in the object in which the multiple types of ultrasonic contrast agents exist.
